(19) United States
(12) Patent Application Publication
(10) Pub. No.: US 2014/0323787 A1
(43) Pub. Date: Oct. 30, 2014

(54) CONCENTRATION SYSTEM AND METHOD

(71) Applicant: Steve Carl, Denver, CO (US)

(72) Inventor: Steve Carl, Denver, CO (US)

(21) Appl. No.: 14/259,864

(22) Filed: Apr. 23, 2014

Related U.S. Application Data
(60) Provisional application No. 61/816,073, filed on Apr. 25, 2013.

(51) Int. Cl. C07C 1/20

(52) U.S. Cl. CPC C07C 1/20
USPC 585/733; 422/187

(57) ABSTRACT

A concentration system and method is disclosed. An example concentration system includes at least two material tubes configured to receive organic material. The example concentration system also includes a collection tank connected to at least one material tube at a time. The example concentration system also includes a recovery unit connected to the collection tank. The example concentration system also includes a solvent charging tank connected to the recovery unit. Under heat and pressure, an extraction from the organic material in the at least one material tube is removed and the solvent is converted into a gaseous state for collection as a liquid solvent in the solvent charging tank.

### PRIORITY CLAIM

This application claims the benefit of U.S. Provisional Patent Application No. 61/816,073 filed Apr. 25, 2013 titled “Concentration System and Method” of Steven A. Carl, and is hereby incorporated by reference in its entirety as though fully set forth herein.

### BACKGROUND

Plant material may include oils and/or other components which may be extracted and used for any of a variety of purposes, including but not limited to extracting essential plant oils for use in, e.g., medicines and lotions.

### BRIEF DESCRIPTION OF THE DRAWINGS

FIG. 1 is an exploded view of various components of an example concentration system.

FIG. 2 is an exploded view showing example components of a material tank of the example concentration system.

FIGS. 3A and 3B show an example top end cap of the material tank shown in FIG. 2, wherein FIG. 3A is a cross sectional view taken along lines A-A in the view shown in FIG. 3B.

FIGS. 4A and 4B show an example bottom end cap of the material tank shown in FIG. 2, wherein FIG. 4A is a cross sectional view taken along lines B-B in the view shown in FIG. 4B.

FIG. 5 is an exploded view showing example components of a collection tank of the example concentration system.

FIGS. 6A, 6B, and 6C are detailed views of the collection tank, wherein FIG. 6A is a front view, FIG. 6B is a top view, and FIG. 6C is a side view.

FIGS. 7A and 7B illustrate the example concentration system as it may be mounted on an example frame.

FIGS. 8A, 8B, 8C, and, 8D illustrate other example frames for the example concentration system.

### DETAILED DESCRIPTION

A concentration system and method for extraction of essential oils from plant material is disclosed. In an example, the concentration system is a closed system, and the systems and methods enable the safe use and active recovery of volatile gas(es) (e.g., butane, as a non-polar solvent). For purposes of illustration, the system and method may be used for the extraction of essential oils. Any plant that contains terpinoids is viable to be used in this system, such as lavender. The system and method are not limited to use in any particular industry, and indeed may have application in any of a variety of fields for use with any of a variety of different organic materials.

An example of the concentration system includes at least one material tank, a collection tank, an induction heater (or other heating element), a recovery unit, and a solvent charging tank. In an example, a pressure sensor is provided adjacent the recovery unit to control the induction heater. A second pressure sensor may be provided on the recovery unit for monitoring and/or controlling operation if the recovery unit's operation is not controlled by an internal pressure sensor. The concentration system may also include multiple tubes, valves, and hoses to enable full or near-full recovery of the solvent, as the solvent evaporates from the plant material. A vacuum pump may also be provided, e.g., to purge the system prior to use.

Before continuing, it is noted that as used herein, the terms “includes” and “including” mean, but are not limited to, “includes” or “including” and “includes at least” or “including at least.” The term “based on” means “based on” and “based at least in part on.”

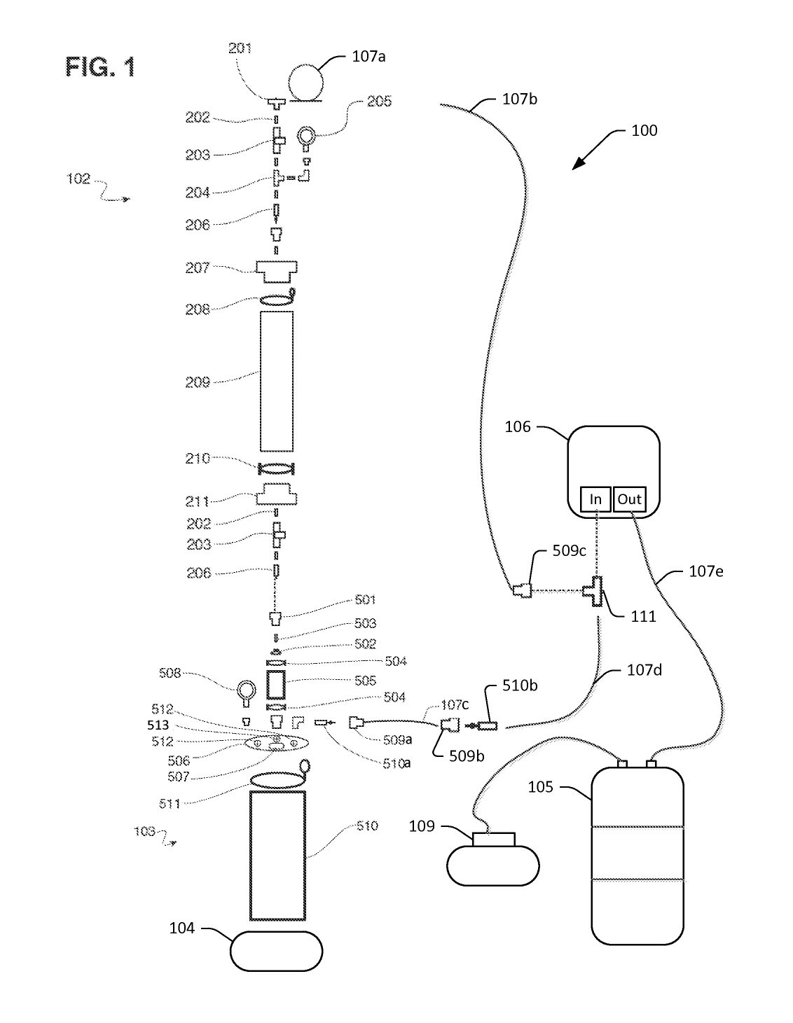

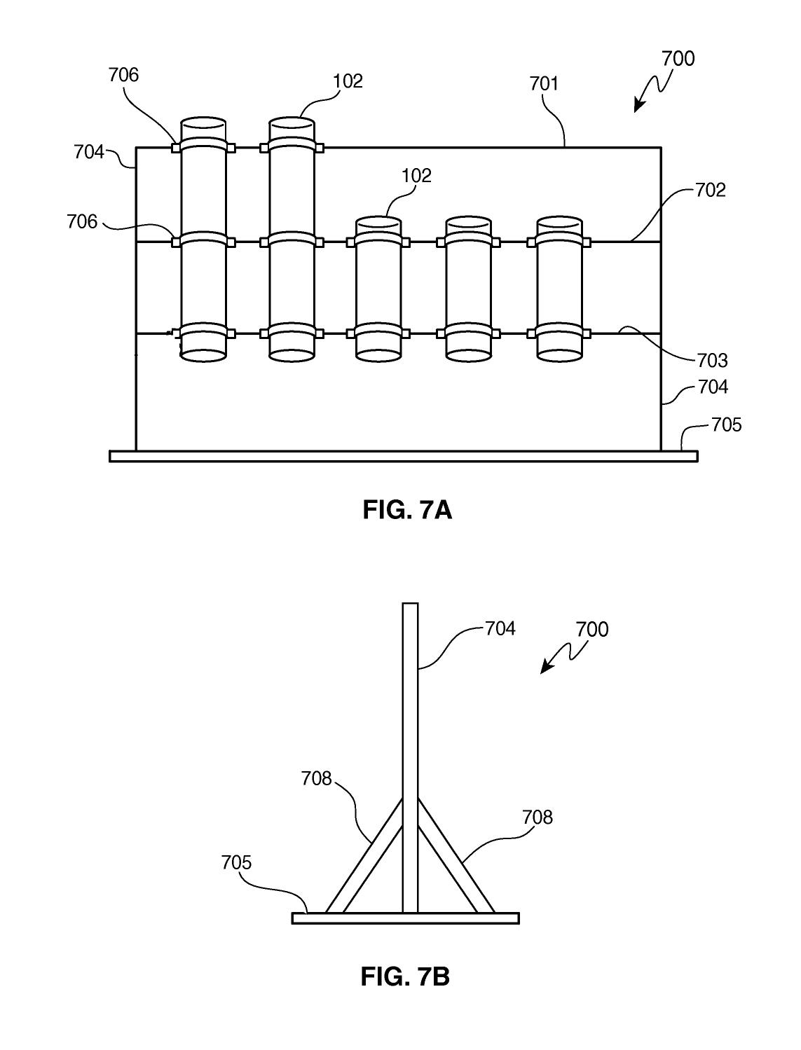

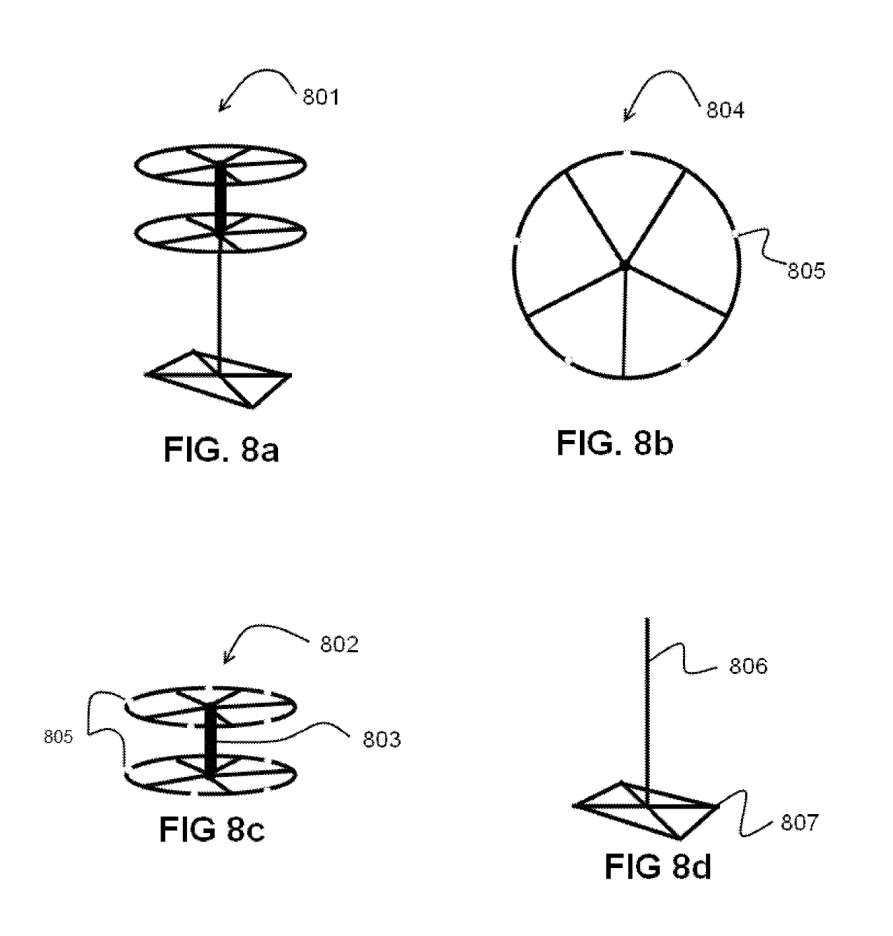

FIG. 1 is an exploded view of various components of an example concentration system 100. Although not shown in FIG. 1, the example concentration system 100 may be attached to a wall or other vertical surface. Of course, the concentration system 100 is not limited to any particular type of mounting, and may also, for example, be provided on a stand with a base constructed to hold the system in a stand-alone configuration as illustrated in FIGS. 7A and 7B and 8.

With reference again to FIG. 1, the example concentration system 100 may include a material tank 102. It is noted that while only one material tank 102 is illustrated in FIG. 1, a plurality of material tanks 102 may be provided and interconnected at the top via hose 107a. In an example, the material tank 102 is constructed of 304 stainless steel. The material tank 102 may have a 4 inch diameter and is 21 or 44 inches long (although other dimensions are also contemplated). The size (21 or 44 inch length) may be selected depending on the amount of plant material to be processed in a batch.

The material tank 102 may be connected to a collection tank 103. In an example, interconnections (not only with the material tank 102, but throughout the system 100) may include hoses, fittings (e.g., stainless 304 steel), quick-connects (e.g., dry-break), and seals (e.g., constructed of Teflon, Viton or Buna-N).

In an example, the collection tank 103 may be implemented as a stainless steel vessel (e.g., manufactured by Apache Stainless Equipment Corporation). The lid of collection tank 103 may have four openings, a one-half inch inlet with a dipstick 513, two ⅜ inch NPT outlets 512, and a one and two inch Tri-Clamp ferrule 507 to be used with a processing view window (e.g., with built in light). The inlet may be fitted with a quick connect/disconnect to enable material tanks to be connected and removed. One outlet may be fitted with a reducer and pressure gauge. The other outlet may be fitted with a 90 degree elbow and three-eighths inch quick connected/disconnect.

A heating element 104 may be located under the collection tank 103 for heating the contents of the collection tank 103. Heating may help to evaporate solvent from the oils extracted from the plant material. The heating element 104 and collection tank 103 can be moved from side to side, to line up with each material tube 102 individually. In another example (see, e.g., FIGS. 8A-D), there is no need to move the collection tank and heating element.

In an example, the induction cooker or heating element 104 may be implemented using a commercially available precision induction cooktop. This cooktop may be digitally controlled and may include an on-off switch (manual or automatic).

The example concentration system 100 may also include a solvent recovery unit 106 connected to the collection tank 103. The recovery unit 106 receives (and may also cool) solvent from the collection tank 103.

In an example, the recovery tank 105 may be implemented using a commercially available Caresaver Universal™ or other oil-less refrigerant recovery pump suitable for R-600 recovery (e.g., Butane). The recovery unit 106 condenses butane gas into a liquid while pumping the liquid back into the recovery tank 105. The inlet may be fitted with two one-quarter inch flares to attach to a pressure sensor (not shown), e.g., via a braided stainless steel hose with quick connect, and to the inlet end of collection tank 103 via connection 509 and hose 107c.

A solvent charging tank 105 receives cooled solvent from the recovery unit 106 and supplies the solvent to the material tank 102. In an example, the charging tank 105 may be used to retain the solvent(s) extracted from the plant material. The charging tank 105 may include a liquid valve with a dipstick used as an outlet, and a vapor valve used as an inlet. The inlet may be attached to the recovery unit 106 via a braided stainless steel hose 107e.

Pressures sensor(s) may be provided (e.g., attached to a support frame and connected using one-quarter inch Ts with flare fittings). For example, pressure sensors may monitor the recovery unit 106. The pressure sensor(s) may indicate when to turn on the recovery unit 106 and/or heating element 104, e.g., enabling operation to start at substantially the same high pressure, while enabling turn-off at different low pressures. Turn-on and/or turn-off may be manual, or partially or fully automated.

In an example, a vacuum pump 109 may be provided. The vacuum pump 109 can be operated to evacuate air from all or part of the system 100 prior to introduction of solvent to the material tanks 102.

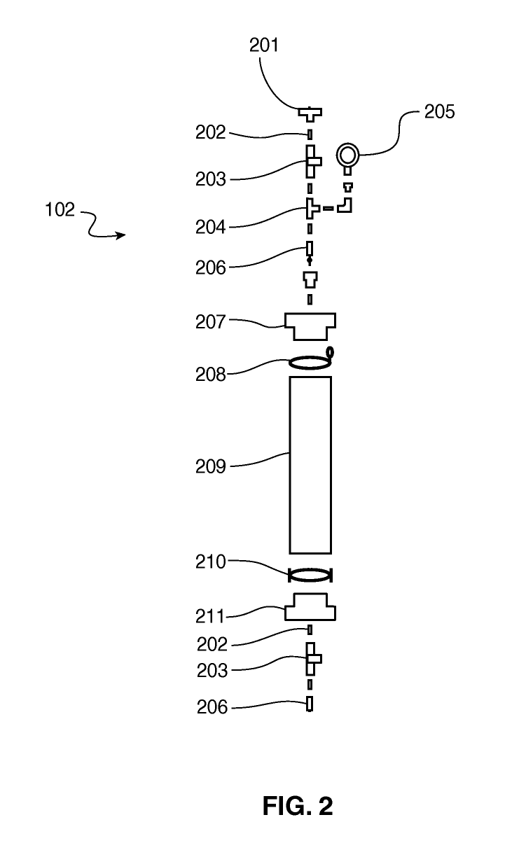

FIG. 2 is an exploded view showing example components of a material tank 102 of the example concentration system 100. In an example, a braided stainless steel hose 107a may be connected into a top cap fitted with a T 201, nipple 202, valve 203 (e.g., a ball valve), a T-joint 204 which leads to a pressure gauge 205, a disconnect system 206 (e.g., a quick disconnect), and a top end cap 207 for the material tank 102.

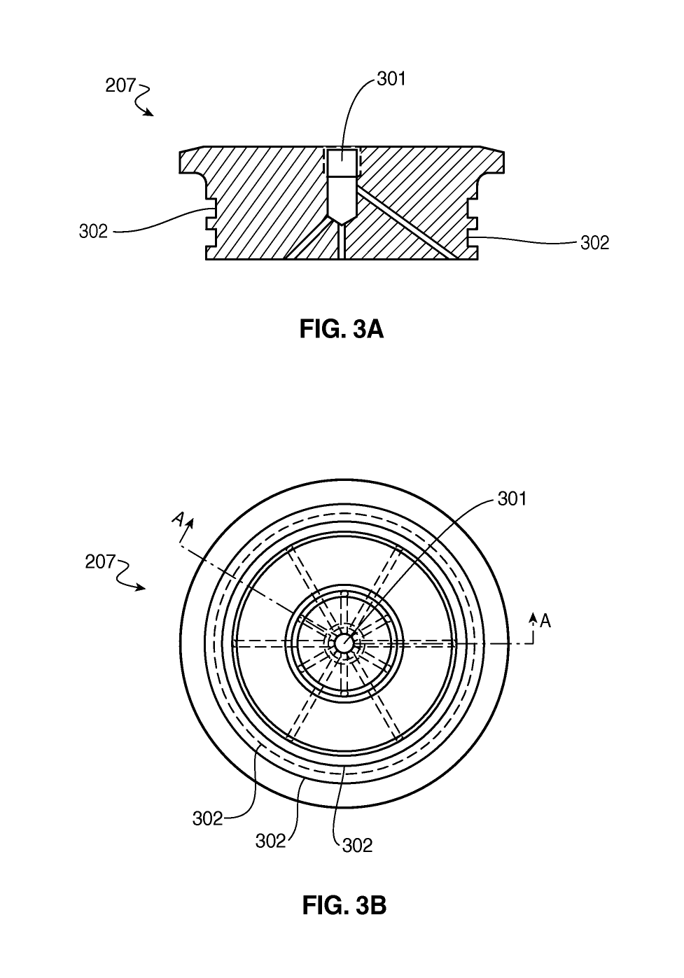

In an example, the stainless steel hoses 107 may fit to a top T 201 on the top of the material tube 102 as shown in FIG. 2. The cap 207 may have one one-quarter inch opening with NPT thread. Ball valves 203 may also be provided at the top and bottom to open and/or close each tube separately. The braided stainless steel hoses 107 are used to transfer solvent between components. The ends may be fitted with quick disconnects 206 to enable quick and easy connection/disconnection. The top end cap 207 is illustrated in more detail in FIGS. 3A and 3B.

In an example, a clamp 208 (e.g., a tri-clamp) connects the top end cap 207 to a tank 209. The tank 209 may be similar to a housing used for filtration (e.g., reverse osmosis which uses high pressure to overcome osmotic pressure). Another clamp 210 (e.g., a high-pressure tri-clamp) connects the tank 209 to a bottom end cap 211. Another valve 203 (e.g., a ball valve), nipple 202, and connection system 206 (e.g., a quick disconnect) connect the material tank 102 to the collection tank 103.

In an example, the bottom fitting or “cap” 211 on the bottom of the material tube 102 may be machined aluminum (or other material), fitted with a quick connect to enable quick connection and removal to the collection tank 103. The bottom cap may include 400 micron screens (or other suitable size), which may be held in place by O-rings. The O-rings may also seal the top end cap 207 of the material tubes 102. The top fittings for the material tubes 102 may include a T-fitting and a direction connection. The bottom cap 211 may be machined aluminum or material of similar function.

FIGS. 3A and 3B show an example top end cap 207 of the material tank 102 shown in FIG. 2, wherein FIG. 3A is a cross sectional view taken along lines A-A in the view shown in FIG. 3B. FIG. 3B is a bottom perspective view of the top end cap 207 showing the side that fits into the interior of the tank 209. In an example, a center aperture 301 is provided to receive the hose 107 and fittings as described above with reference to FIG. 2. In an example, grooves 302 may receive O-rings to provide a secure fit for the top end cap 207 to the tank 209.

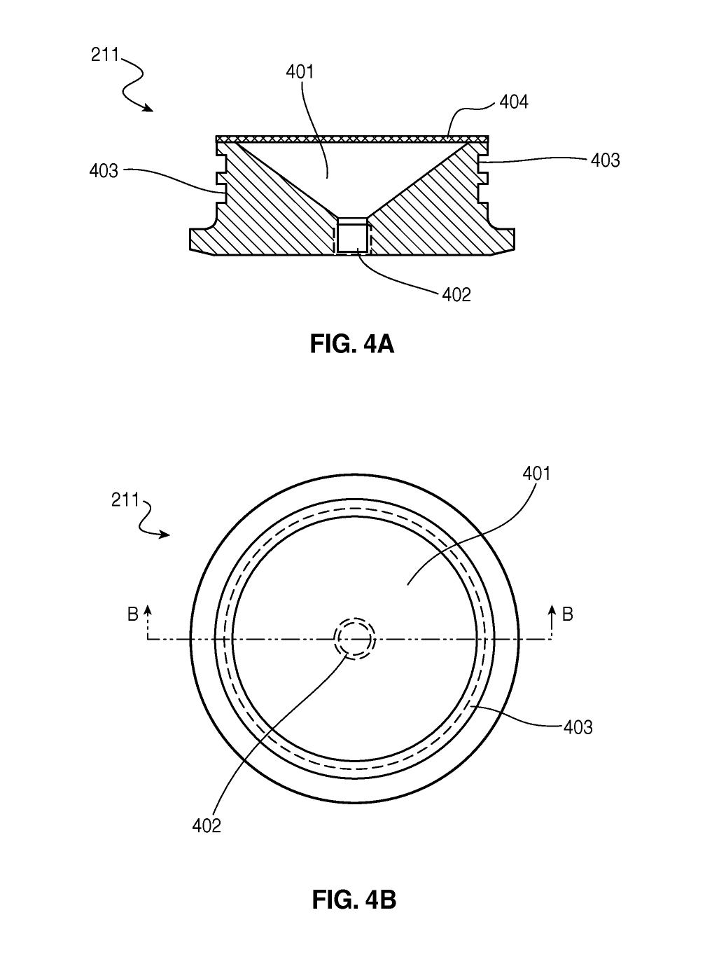

FIGS. 4A and 4B show an example bottom end cap 211 of the material tank 102 shown in FIG. 2, wherein FIG. 4A is a cross sectional view taken along lines B-B in the top view shown in FIG. 4B. FIG. 4B is a top perspective view of the bottom end cap 211 showing the side that fits to the interior of the material tank 102. As seen in FIG. 4A there is a funnel-shaped opening 401 ending in a one-quarter inch NPT thread 402 at the center. The smaller aperture 402 connects to a quick connect that leads to the collection tank 103.

In an example, grooves 403 may receive O-rings to provide a secure connection of the end cap 211 to the tank 209. A filter 404 (e.g., screen or mesh) fits over the opening 401 and serves to prevent plant material from entering in the tank 209.

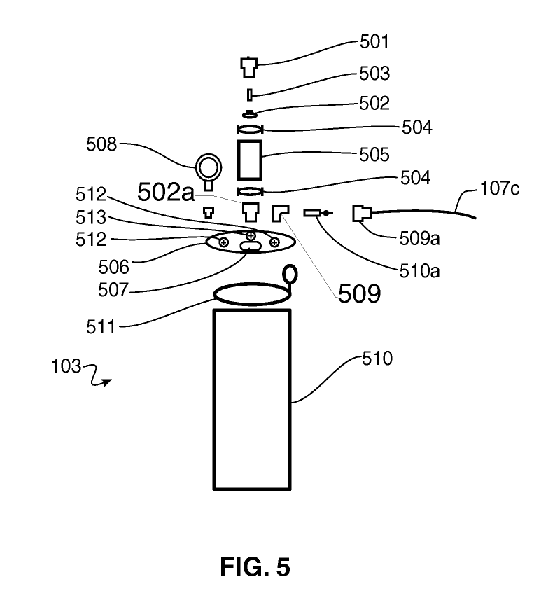

FIG. 5 is an exploded view showing example components of a collection tank 103 of the example concentration system 100. A connection 501 (e.g., quick disconnect) receives the quick connect 206 from the material tank 102. A nipple 503 fit to a one and one half inch tri-clamp, one-quarter inch NPT adapter 502, fitted to a sight glass 505 via a clamp 504 (e.g., a high pressure tri-clamp). The sight glass 505 is fitted to a one and one half inch tri-clamp, one-half inch NPT adapter 502a. The adapter 502a is connected to the inlet 513 of the collection tank 103.

In an example, a top of collection tank 506 is provided with a pressure gauge 508. A 90 degree elbow fitting 509 and quick disconnect 510a secure the outlet hose 107c for connection via fitting 509b (see FIG. 1) and quick disconnect 510b (see FIG. 1) and hose 107d to recovery unit 106, e.g., via T-connector 111.

The top of the collection tank 506 may be fitted to the collection chamber 510 with a clamp 511 (e.g., an eight inch tri-clamp). Openings 512 in the top of the collection tank 506 provide inlets from the material tank 102 to the pressure gauge 508. Openings 512 may also be configured as an outlet to the recovery unit 106 via hose 107c and 107d.

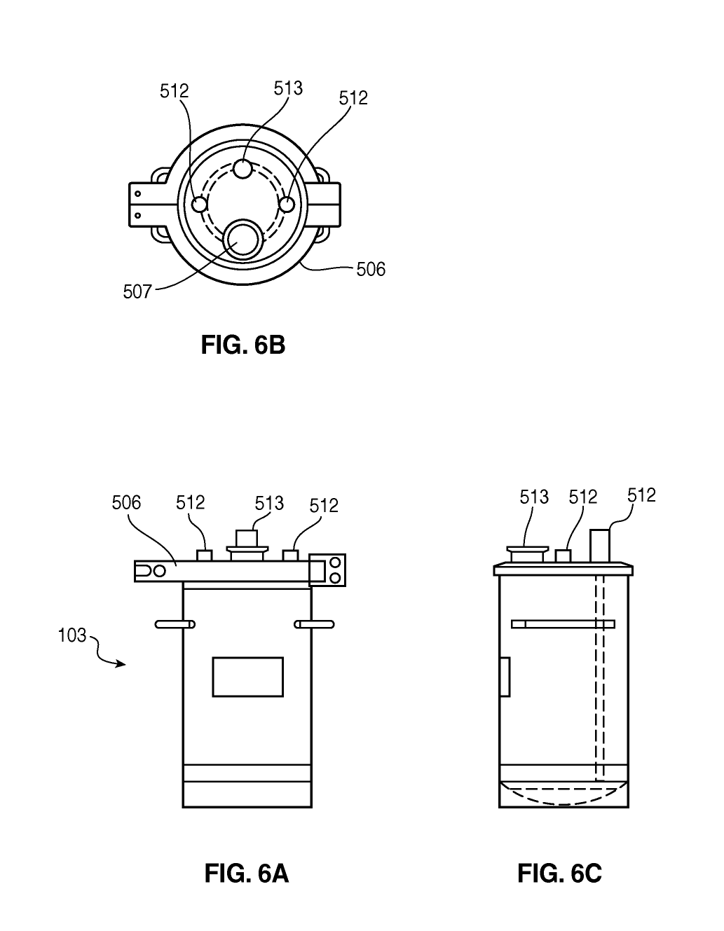

An example of the assembled collection tank 103 can be seen in FIGS. 6A, 6B, and 6C. FIGS. 6A, 6B, and 6C are detailed views of the collection tank 103, wherein FIG. 6A is a front view, FIG. 6B is a top view, and FIG. 6C is a side view.

FIGS. 7A and 7B illustrate the example concentration system 100 as it may be mounted on an example frame 700. FIG. 7A illustrates a front view of the frame 700 with material tanks 102 of two different sizes. In an example, the frame 700 has at least two crossbars to support the smaller size material tank 102 (three are shown in FIG. 7A). In an example, the crossbars are positioned such that a range of size of material tanks 102 as shown, including but not limited to a 21 inch tank or 44 inch tank, can be fitted to the frame.

In an example, the frame 700 has a top crossbar 701, and a bottom crossbar 702, fitted to two uprights 704. A base 705 supports the uprights 704. The crossbars 701, 702, 703 support the material chambers 102 via quick release clamps 706. The quick release clamps 706 can be easily opened for material tank to be removed or slid op and down to connect to collection tank 103, or closed to hold in place.

FIG. 7B illustrates a side view of the example frame. The base 704 is sufficiently sturdy and wide to support the material tanks 102 without tipping. In an example, one or more support bars 708 connect the base 705 to each of the uprights 704. The frame 700 may be constructed from a one inch steel tube welded together. The frame 700 may have at least two vertical side rails or crossbars 701, 702 welded to the uprights 704.

FIGS. 8A, 8B, 8C, and, 8D illustrate other example frames that enable the material tanks to be mounted to a swivel, making it possible to switch the material tanks without moving the collection tank and heating element. FIG. 8A is an example of the optional frame in its entirety. FIG. 8B is the circular frame 804 that has a diameter of about twelve inches; with holes 805 placed at even intervals to allow quick release clamps 706 to allow material tanks 102 to be attached. FIG. 8C is an example of a complete spindle apparatus with two circular frames 804 welded to a twelve inch tall, hollow tube with a diameter large enough to allow it to slide on top of the vertical support 806 of the frame. FIG. 8D illustrates the support frame with a base 807 that is about thirty inches by thirty inches with support beams welded around the edge and a vertical support 806 that is about fifty-two inches tall.

Before continuing, it should be understood that the example components described above with reference to the figures are intended only to be illustrative of an example concentration system. The concentration system is not intended to be limited to any specific type, number, or configuration of components.

The concentration system 100 may be operated according to the following example protocol. It is noted, however, that these operating parameters are provided only by way of example, and the protocol should not be construed to be limiting in any way. It is also noted that, one or more of the processes may be automated and/or partially automated (e.g., using computer controls, sensors, and/or robotics).

In an example, an operator may remove the top end cap 207 from the material tube 102 (e.g., by using a tool such as a flat-head screwdriver to pry open the top end cap 207). The material chamber 102 may be filled (or partially filled) with plant and/or other organic material. The “loading” material can be packed in tight, as desired. For example, a 21 inch tube may hold up to about 500 grams of plant material, and a 44 inch tube may hold up to about 900 grams of plant material.

In an example, several drops of oil (e.g., vegetable glycerin) may be added on the O-ring fittings of the top cap and spread all the way around the ring for an air-tight seal and to provide a lubrication effect (e.g., allowing easy assembly and removal). The top end cap 207 is then pushed back into place on the material tube 102, and the band or tri-clamp 208 that holds the top end cap 207 is replaced on the tube 209 and tightened by hand.

The collection tank 103 and heating element 104 (e.g., induction cooker or other heating element) may then be placed under the material tank 102. In an example, the operator makes sure the heating element 104 is directly under the center of the collection chamber 103 for even heating.

In an example, quick release clamp 706 may be released while holding the material tank 102. Slowly lowering the material tube 102 enables the quick connect 206 to slide together between the bottom end cap 211 of the tube 209 and the collection tank 103. A hose 107c may be connected between the outlet on the collection tank 103 opposite the pressure gauge 508 to the inlet of the recovery unit 106 via connection hose 107d using the connection(s) 509b and 510b.

Ball valves on the top of the material tubes 102 may be turned on or opened, and the tri-clamp 511 holding the lid 506 of the collection tank 103 tightened. When all fittings have been made, the vacuum pump 109 may be connected to top hose assembly 107b via quick disconnect fitting 509c. After making sure all valves on material tanks not in use are off or closed, the vacuum pump 109 can be turned on, and brought to about negative 28 pounds per square inch (PSI). The vacuum pump 109 removes the air from the system before use to reduce or altogether eliminate risk of fire. Once the desired pressure is achieved the ball valve 203 on the top of the material tank 102 in use is closed.

The vacuum may also be implemented to reduce pressure in the charging tanks. In an example, the vacuum is configured as a 3 cubic feet per minute (CFM) vacuum, and may be fitted with a quick connect. The hose 107b may then be removed from the vacuum pump 109, and attached to the recovery tank 105 for operation. The vacuum pump 109 may then be turned off, and the ball valve between the material tubes 102 and the collection tank 103 turned off.

The recovery tank 105 sits on a charging scale. In an example, a charging scale (not shown) may be implemented as a commercially available YELLOW JACKET™ model #68802. The charging scale measures the amount of solvent already in the system prior to use, and the amount of solvent recovered during and after operation.

The charging scale may be turned on and tarred. The liquid valve of the charging tank 105 is opened, and the ball valve (of the top of the material tube 102) is opened to allow liquid solvent to flow into the plant material.

The operator may watch the scale and close the liquid valve of the charging tank 105 when the desired amount of solvent has been collected in the material tube 102. In an example, a ratio of about 0.25 grams of solvent can be collected for each gram of plant material. This ratio may vary based on quality of the plant material, operating environment, and/or other characteristics.

During operation, the plant material is allowed to “soak” (based on amount of plant material in the system, or as desired (e.g., for at least about 5 to 20 minutes or more). A vapor valve of the charging tank 105 may be opened and connected to the outlet of the recovery unit 106 (e.g., with hose 107e).

The recovery unit 106 is turned on and is be controlled by the pressure sensor. The ball valve between material tube 102 and collection tank 103 is opened to allow the solution (now containing the solute) to flow into the collection tank 103 (e.g., through a 400 micron steel mesh screen that helps prevent the plant material from being removed from the material tube 102).

In an example, pressure sensor(s) may be implemented as a feedback to a switch to automatically cause the heating element 104 to turn on at about 9 PSI, and the recovery unit 106 to turn on at about 10 PSI. The induction cooker turns on when power is sent from the pressure sensor. The heating element 104 heats the solution to about 100 degrees Fahrenheit and boils the solvent, thus turning the solvent into a gas.

The collection tank 103 may be heated to speed-up transformation of the solvent from a liquid to a gas, which the recovery unit 106 then pumps back into the charging tank 105. The heating element 104 can be manually turned off or automatically turned off by the pressure sensor at 7 PSI (adjustment of this range may be necessary due to environmental conditions). The recovery unit 106 can be manually turned off or automatically turned off at or just under zero PSI by the pressure sensor. Once the recovery unit has turned off, the top and bottom ball valves 203 on the material tank 102 can be shut off (e.g., automatically or manually). The material tank 102 can then be removed from the collection tank 103 by disconnecting the quick disconnect 501 and sliding the material tank 102 up and re-securing it to the frame via clamp 706.

The collection tank 103 can now be opened by first removing the tri-clamp 511, then the lid. The extracted material can now be removed from the collection tank 103 and the final purge of remaining solvent can be performed per operator's preference.

The material tube 102 (with valves on the top and bottom closed) starts to build pressure as the solvent evaporates from the plant material. The next material tube 102 is ready to be used. When all of the material tubes 102 have been used and the ball valves are closed on both ends, the hose 107b can be removed from the charging tank 105 and attached to the inlet of the recovery unit 106 via quick disconnect 509c, hose 107d, and quick disconnect 510b.

The ball valves 203 at the top of the system can now be opened, and the recovery unit removes the gaseous solvent as it evaporates from the plant material. This may take a couple of runs to fully complete. The operation is complete when the pressure gauges 205 at the top of the material tubes 102 stay at or about zero.

The heater may be turned off (e.g., automatically and/or manually based on reading the pressure sensor) when the pressure is about 5 PSI. The recovery unit 105 may be turned off (e.g., automatically and/or manually based on reading the pressure sensor) when the pressure is about 0 PSI.

During operation, the recovery unit 106 pumps gas out of the collection chamber 103, turning the gaseous solvent into a liquid and returning it to the charging tank 105.

When the recovery unit 105 is shut off, the ball valves on the bottom and top end caps of the material tube 102 are also shut off (e.g., automatically and/or manually). The material tube 102 can be moved away from the collection tank 103 and secured with the quick release clamp 706 attached to the frame 700. The hose 107 connected to the collection tank 103 may be detached, and the collection tank 103 removed from the heating element 104. When all the solvent has been removed from the material tanks 102 via the recovery unit 106, as indicated by the pressure gauges 205 retaining a value of 0 or just under 0 PSI, the end caps may be opened and the extract removed. For example, the top end caps can be removed, and the spent plant material can be vacuumed out of the material tubes 102 using a shop vacuum.

An example adaptation to the system includes separating unwanted materials from the solution and collection (e.g., using a Buchner funnel). The example includes providing an empty 21 inch material tank as a primary winterization chamber. By adding a Buchner funnel in between the screen 404 and the bottom end cap 211, it is possible to run a material tank normally into the empty 21 inch tank. Then, the solution can be frozen (e.g., at sub-zero temperatures). Next, the material tank can be attached to the frame and run into the collection chamber.

The yield from the system is based at least in part on the type, quantity, and/or quality of plant (and/or other organic material used). In an example, an average yield was realized of between about 10-20% by weight of the original plant material. The potency of the final product is also dependent at least to some extent on the plant material characteristics, the purging technique(s) used, and whether a second solvent is utilized (e.g., outside of the system). For example, potency can range for example between 50-95% essential plant oils.

It is noted that the examples shown and described are provided for purposes of illustration and are not intended to be limiting. Still other examples are also contemplated.

1. A concentration system comprising:
at least two material tubes configured to receive organic material;
a collection tank connected to the at least one material tube;
a recovery tank connected to the collection tank via recovery unit; and
a solvent charging tank connected to the recovery tank via a recovery unit, wherein under heat and pressure, an extraction from the organic material in at least one material tube is removed and the solvent used is converted into a gaseous state for collection as a liquid solvent in the solvent charging tank.

2. The concentration system of claim 1, further comprising a heating element to convert the extraction from the organic material to the gaseous state.

3. The concentration system of claim 1, further comprising a pressure sensor configured to automatically control operation of the recovery unit, if one is not built into said unit.

4. The concentration system of claim 1, further comprising a pressure sensor configured to automatically control operation of a heating element.

5. The concentration system of claim 1, further comprising a vacuum pump.

6. The concentration system of claim 1, further comprising a charging scale operatively associated with the charging tank.

7. The concentration system of claim 1, wherein at least two material tanks are fitted with end caps on each end to form a vacuum tight seal.

8. The concentration system of claim 7, further comprising a screen fitted to at least one of the end caps.

9. The concentration system of claim 7, further comprising funnel shape formed on at least one of the end caps.

10. The concentration system of claim 1, wherein the liquid solvent is butane.

11. A concentration system comprising:
at least two material tubes configured to receive organic material;
a collection tank connected by a first hose to at least two material tubes;
a collection tank connected by a second hose to the recovery unit;
a solvent charging tank connected by a third hose to the recovery unit; and
at least one pressure sensor to automatically start and stop extraction operations, wherein under heat and pressure, an extraction from the organic material in the at least one material tube is removed and the solvent is converted into a gaseous state for collection as a liquid solvent in the solvent charging tank.

12. The concentration system of claim 11, further wherein at least one pressure sensor is configured to automatically control operation of a heating element.

13. The concentration system of claim 11, further wherein at least one pressure sensor is configured to automatically control operation of the recovery unit.

14. The concentration system of claim 11, further comprising a heating element to convert solvent to the gaseous state.

15. The concentration system of claim 11, further comprising a vacuum pump connected to the entire system.

16. A concentration method, comprising:
receiving plant material in at least one material tube;
collecting a solution from at least one material tube in a collection chamber; and
heating the solution in the collection chamber to produce a gaseous solvent.

17. The method of claim 16 further comprising cooling the gaseous solvent to form a liquid solvent.

18. The method of claim 16 further comprising sensing pressure to control the heating and cooling steps.
